(19) United States
(12) Patent Application Publication
(10) Pub. No.: US 2003/0077572 A1
(43) Pub. Date: Apr. 24, 2003

(54) Quantitative differential display using isotope ratio monitoring

(76) Inventors: Fred P. Abramson, Washington, DC (US); Paolo Lecchi, Rockville, MD (US)

Correspondence Address: ANTONELLI TERRY STOUT AND KRAUS, SUITE 1800, 1300 NORTH SEVENTEENTH STREET, ARLINGTON, VA, 22209

(21) Appl. No.: 10/268,669

(22) Filed: Oct. 11, 2002

Related U.S. Application Data
(60) Provisional application No. 60/328,083, filed on Oct. 11, 2001.

(51) Int. Cl. C12Q 1/00; G01N 30/02; C12N 15/85

(52) U.S. Cl. 435/4; 435/455; 436/161

(57) ABSTRACT

Disclosed are methods for quantitatively monitoring components of a cell population, for quantitatively monitoring a difference between components of first and second samples of respective cell populations growing in different conditions, and/or for tagging moieties in biological molecules, and apparatus for performing these methods. Each method and apparatus labels at least a first population of cells with stable isotopes, combines the labeled cells with control cells, and detects isotopic enrichment using an isotope ratio-monitoring detector. The isotope ratio-monitoring can be performed using chemical reaction interface mass spectrometry.

This application claims priority under 35 USC 119(e)(1) of Provisional Application No. 60/328,083, filed Oct. 11, 2001.

The present invention is directed to a method for quantitatively monitoring a cell population, using an isotope ratio-monitoring detector, and apparatus for performing this method. In one aspect of the invention, the present invention is directed to a method for quantitatively monitoring a difference between cell populations growing in different conditions, and apparatus for performing this method, which can show what is different between the two cell populations while ignoring their common features, and permits determining how much difference there is between the two cell populations.

Several reports, listed in the following, describe the use of stable isotope enrichment to define expression levels of proteins, and these reports are incorporated herein by reference in their entirety:

1. Pasa-Tolic et al., J. Am. Chem. Soc. 121:7949-7950 (1999)

2. Oda et al., Proc. Natl. Acad. Sci. 96:6591-6596 (1999)

3. Gygi et al., Nature Biotechnology 17:994-999 (1999)

4. Mann, ibid 954-955

5. Veenstra et al., J. Am. Soc. Mass Spectrom. 11:78-82 (2000)

However, the strategy in these reports requires some critical assumptions about which, e.g., proteins are to be investigated. Indeed, with conventional mass spectrometric approaches, an operator must focus on the mass of the protein of interest, thus requiring an assumption about which protein or proteins to choose. Computer algorithms have been developed to automate the process, but the ability to pick out the appropriate species is not robust.

Against this background, the present invention uses stable isotope labeling and isotope ratio monitoring to avoid the need for the critical assumptions. In particular, the present invention has uniform labeling of cells, in combination with isotope ratio monitoring. This combination allows detection and quantification of substances biosynthesized in different concentrations under different conditions in a general manner, that does not depend on any operator assumptions about “what to look for”.

In particular, the present invention provides a method, and apparatus, for quantitatively monitoring a cell population, including labeling (e.g., uniformly labeling) a population of cells with stable isotopes; mixing the cells labeled with the stable isotopes, with control cells grown without being labeled with the stable isotopes, so as to form a mixture; and detecting resulting isotopic enrichment of the mixture, using an isotope ratio-monitoring detector.

Isotope ratio monitoring is a process whereby all species being examined are decomposed to small, common species before passing into an isotope-selective detector. Isotope ratio monitoring can be a continuous process, usually involving some type of chromatography, whereby isotopic, rather than structural, information about each species can be obtained. Isotope ratio monitoring is usually done with mass spectrometry, but other forms of spectroscopy can be used if suitably configured to continuously accept and analyze samples.

A specific type of isotope ratio-monitoring detector is the CRIMS (chemical reaction interface mass spectrometry), a technique for isotope ratio monitoring developed by one of the present inventors (Dr. F. Abramson) and described, for example, in U.S. patent application Ser. No. 09/023,481, filed Feb. 13, 1998, the contents of which are incorporated herein by reference in their entirety. CRIMS can also provide selective element detection for certain biologically interesting elements.

With isotope ratio monitoring (using CRIMS or another method of converting a range of analytes to common products), the substances under investigation (for example, and not to be limiting, proteins) are all decomposed to a common set of small molecules that give a continuous record of the isotope ratio while monitoring a single set of masses. All enriched materials are detected with the mass spectrometer set in a continuous configuration.

Isotope labeling can be done in many ways. As known in the art, one or more isotopically labeled species are incorporated into the desired molecules by chemical means. Such incorporations can also been done biologically, using cells or animal systems. Illustratively, and not to be limiting, the labeling could take place in cells in a living species (e.g., animal or human), and in this case, for example, labeling can be accomplished by feeding or administering labeled nutrients that become incorporated in the class of biological molecules being studied.

Only occasionally has uniform labeling been attempted. With uniform labeling, all atoms of the desired molecular species contain an identical fraction of the desired isotope, according to aspects of the present invention. While this also can be done chemically, for anything other than the simplest biological molecules such products more likely arise from growing cells in the presence of a source of, for example, carbon that contains an excess amount of its minor isotope ¹³C. For simple cells, the sole source for carbon can be glucose or even CO₂, as examples. For complex cells, for example, mammalian, a special growth medium containing ¹³C-enriched essential amino acids and glucose can be used. In this way, uniformly and completely labeled cellular products can be prepared, which can then be processed by procedures according to the present invention to achieve the desired results of quantitatively monitoring cell populations.

In general, aspects of the present invention include a technique for quantitative differential display of modifications occurring in a cell population grown in different (e.g., two different) conditions (for example, control versus treated), each of which is associated with a specific isotope ratio (e.g., ¹³C/¹²C). The differential display refers to a process that shows what is different between two samples, while ignoring their common features. Quantitative differential display would permit determining how much difference there is between these samples.

The experimental design according to aspects of the present invention includes the following:

(a) Uniformly labeling an experimental population of cells with stable isotopes (i.e., non-radioactive labels);

(b) Mixing these cells (for example, treated to be uniformly labeled) with control cells grown under standard (non-labeled) conditions; and

(c) Detecting the resulting isotopic enrichment using an isotope ratio-monitoring detector.

The foregoing is described in connection with control cells grown under non-labeled conditions. However, the present invention would also work on two populations of cells, each of which was labeled, but that were labeled to different extents. This is because, in general, the method of the present invention looks at differential labeling, and does not require that one of the two populations be non-labeled. Also, one can “strip” most or all of the minor isotope from a sample (e.g., ¹³C depleted carbon) to generate an isotopic difference from another sample. These possibilities achieve the same results as adding products from labeled cells to products from non-labeled cells, and measuring their isotope ratios.

This analytical procedure allows one to quantitatively monitor any biological molecule from any class of biological molecules that is synthesized more or less extensively in one experiment than in another experiment. For quantitatively monitoring a difference between cell populations growing in different conditions, the following processing can be utilized and forms an aspect according to the present invention.

A first population of cells is labeled with stable isotopes, forming a labeled first population. A second population of cells is labeled with stable isotopes, and a stimulus or variant of the original cell is also provided in the growth condition of this second population, forming a labeled second population. Control cells are combined with each of the labeled first and second populations, respectively to form first and second samples. Thereafter, isotopic enrichment of the first and second samples is detected, using an isotope ratio-monitoring detector, and the resulting isotopic enrichments of these two samples are compared to provide the differential display, and, in particular, the quantitative differential display.

FIG. 1 gives a schematic representation of a concept of differential monitoring of biosynthetic products combining the use of uniform, stable isotope labeling procedures and CRIMS detection (which is a preferred form of isotope ratio-monitoring, although not the only form of monitoring).

Briefly, referring to FIG. 1, which is a scheme of CRIMS for quantitative isotope ratio monitoring using uniformly labeled growth media, separate cultures of cells are grown in parallel using media that differ just in the isotope ratio of ³⁴S/³²S, ¹³C/¹²C, ¹⁵N/¹⁴N, ¹⁸O/¹⁶O or ²H/¹H. With the enriched medium, the cells are grown for sufficient time for them to achieve isotopic equilibrium with the growth medium, typically four or more cycles of division. In this way, any biosynthesized products from the cell will have identical isotope ratios. In one flask (culture of cells), a stimulus (for example, pharmacological, chemical or physical) is applied to the cells or else a variant (genetically modified, diseased, etc.) of the original cell is used, in contrast with another flask of control cells under control conditions. Cells are then harvested and combined at known volume dilutions to produce two samples:

(1) A first sample, having control cells combined with isotopically labeled control cells (that is, a control sample); and

(2) A second sample, which is control cells combined with isotopically labeled cells that have been stimulated/modified (that is, the experimental sample).

The sample (first sample) where both cell incubations were done under control conditions establishes a control amount of enrichment of the mixture, that is used to establish the enriched isotope ratio of analytes with unaltered expression when the experimental cell population is analyzed.

The analysis may involve secreted or intracellular material. Procedures appropriate to obtaining such samples, which are conventional procedures, are used. For example, products may be fractionated into different classes of biological compounds (for example, carbohydrates, proteins, lipids, RNA, DNA, etc.) or separated in subcellular components (for example, nuclei, membranes and mitochondria). Each fraction represents a defined “chemical” or “functional” sub-unit of the organization under evaluation. Chromatographic (for example, high performance liquid chromatography (HPLC)) techniques, as shown in FIG. 1, or electrophoretic techniques are then used to further fractionate these sub-units. Any analyte with an altered expression level in the experimental sample will differ in isotope ratio from the control sample because its concentration is different from its counterpart in the control sample. To monitor the isotope ratio of these analytes, a detector able to measure continuously and accurately the isotopic ratio and consequently changes in expression levels, is connected directly to the separation technique, as shown in FIG. 1. For the analytical scheme developed according to one aspect of the present invention, the monitoring (detection) is a combination between HPLC (separation techniques) and CRIMS (measurement techniques).

As an additional aspect of the present invention, as seen in FIG. 1, a “T” connection is provided between the HPLC and the CRIMS, where some of the effluent from the HPLC diverted for future analysis by, for example (and not to be limiting), an electrospray mass spectrometer (ESI) or any other type of structural analysis.

FIG. 1 also shows the carbon trace from the CRIMS, and the isotope enrichment trace, from each of the two samples, of the monitoring according to the present invention.

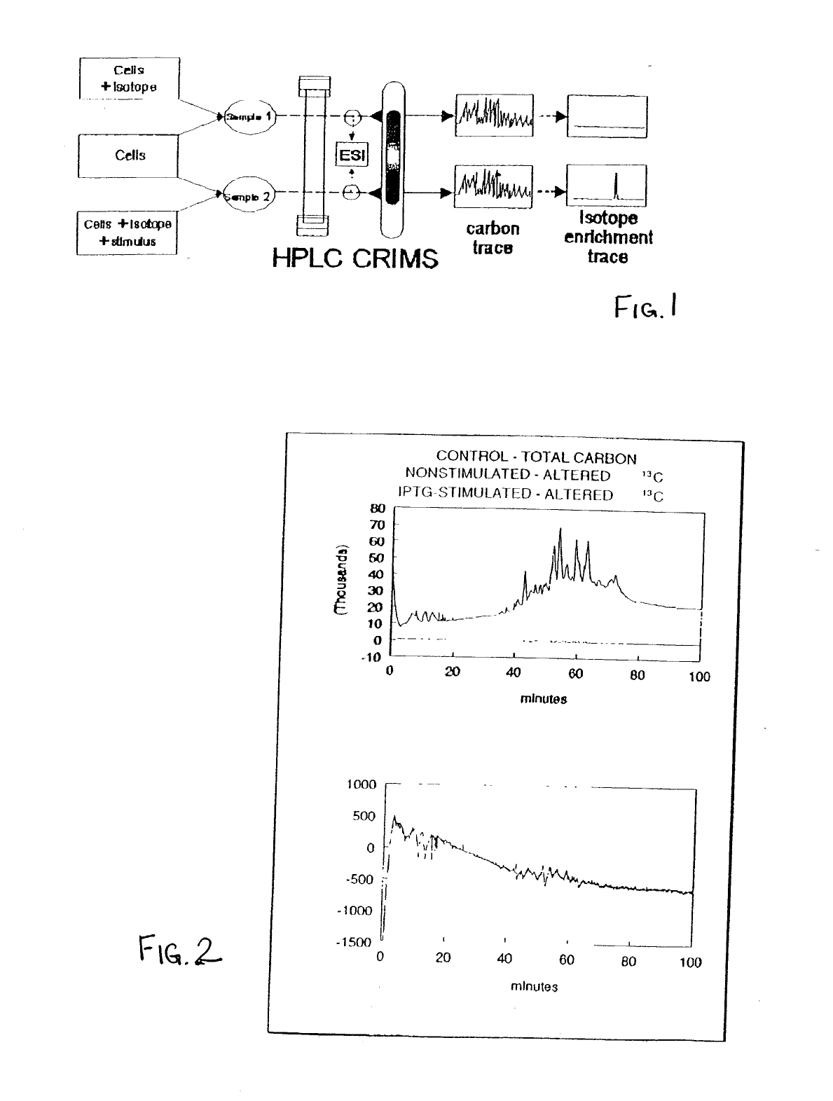

FIG. 2 provides data from an experiment showing quantitative differential display with isotope ratio monitoring, i.e., a control experiment. For providing the results shown in FIG. 2, three specimens of proteins from E. coli were obtained under different conditions: (1) unstimulated using unenriched medium; (2) unstimulated using enriched medium; and (3) stimulated using enriched medium, and were mixed as described above, to yield two samples. The graphs of FIG. 2 show the results from part of this experiment. The top trace monitors essentially all the carbon in the extracted proteins, and its two lines at about zero abundance represent the differential display. The small signals generating nearly straight lines indicate how much selectivity the present invention has against analytes with normal expression. The differential display on an expanded scale (the lower chart of FIG. 2) contains essentially no signals from unstimulating cells.

Attention is also directed to FIG. 3, which is a comparison of differential expression in control and stimulated cells. In this figure, a comparison of differential information from the control cells (top) and the stimulated cells (bottom) is shown. The presence of several peaks is evident here. The upper trace shows that the system is capable of removing all information that is not relevant in measuring differential expression. The lower trace shows those regions of the chromatogram where materials of altered (in this case, elevated) expression occur. The height of each peak relates quantitatively to the excess amount of that species generated by the stimulus. Thus, the lower chart shows quantitative differential display with isotope-ratio monitoring.

In the following is described a method for generating the data shown in FIGS. 2 and 3. To generate this data, a mathematical function is used that subtracts a percentage of the signal for Sample 2, based on the abundance from Sample 1. Illustratively, and not to be limiting, the natural abundance of ¹³C is 0.0117, and this is the expected value for the amount of ¹³CO₂ (measured with the mass spectrometer at mass 45, its molecular weight) compared to ¹²CO₂ (measured at mass 44) that would be obtained in isotope ratio monitoring of unenriched samples. By subtracting 0.0117×the observed signal at mass 44 from the observed signal at mass 45, all the peaks would disappear. If one or more peaks had more ¹³C than 0.0117, that selected group of peaks alone would show up in this mathematically-manipulated observation. In FIGS. 2 and 3, a Sample 1 was used to find a number that, when multiplied with the signal observed at mass 44, subtracted all the peaks in the control sample, to give a straight line as shown in the top half of FIG. 3. This number used in this case was 0.08. Without this manipulation, every peak in the top of FIG. 2 would have to be individually examined for its isotope ratio and compared between Samples 1 and 2, to look for differential expressions. Thus, through use of this subtraction technique, significant differences can easily be seen, and this subtraction feature would be a desirable feature in any commercial data system of the present invention.

As an additional aspect of the present invention, the process and apparatus thereof can be used in tagging specific moieties in biological molecules, by performing an “aimed isotopic labeling”. In this case, the medium contains labeled precursor representing the building block(s) for a specific structure under evaluation. In vivo labeling followed by chromatographic separation and isotopic ratio detection are performed, as discussed previously, having a specific aim of tracing a particular feature within biological molecules. The purpose of this approach is the same used in conventional tracing techniques where radioactive isotopes (that is, ¹⁴C, ³H, ³²P, etc.) are used to tag specific structures in a molecule. Such specific moieties include, as examples, methylation, glycosylation, acetylation, alkylation, nitrosylation, phosphorylation, sulfation, etc. Use of labeled precursors as in this aspect of the present invention, together with the isotope ratio-monitoring device; or just the presence of an unusual element, such as Se, P, S or Cl, and CRIMS or another element-selective device, provides the differential display.

The present technology is best described using the example of proteomics.

A technique used in proteomics must be able to detect and measure changes in a number of proteins from among the wide array of cellular proteins. The general, conventional approach to tackle this analytical problem is to separate the components contained in a protein mixture on a flat gel (two-dimensional gel electrophoresis). The separated proteins are then stained to produce spots of different intensities that are related to the amounts of proteins contained within the spots. The identification of proteins contained in spots of interest can be achieved by mass spectrometry. This strategy has many weaknesses. Most importantly, the definition of “what is the spot (i.e., the protein) of interest” in a two-dimensional gel (or in any type of chromatography) is arbitrary (that is, which of these many proteins should be evaluated?). Many important proteins could be lost by interference from less important, but more abundant, species. Note Gygi, et al., Proc. Natl. Acad. Sci. U.S.A. 2000, 97:9390-5.

In contrast to the foregoing, the approach of the present invention is comprehensive, without involving any operator judgment. Two-dimensional gels are unsuitable for unusual proteins (for example, proteins with a molecular weight of more than 10⁵ Da or less 10⁴ Da). On the other hand, CRIMS has a nearly unlimited mass range. The dynamic range of detection of gels is low, probably less than 10-fold, so that those proteins with an intrinsically low level of expression will not be seen. In contrast, with CRIMS, accurate isotope ratio measurements over at least three orders of magnitude of concentration can be made.

The two-dimensional gel cannot separate more than 700-800 proteins, which represent just a fraction of the proteins potentially expressed by an organism. Using the stable isotope ratio-monitoring method, the requirement for exceptional chromatographic resolution is relaxed. The simplicity of the lower chromatogram in FIG. 3 is compared with the complex appearance of the top chromatogram in FIG. 2. With CRIMS, a labeled protein can be distinguished from an unlabeled material even when the unlabeled species is present at 1,000-fold excess. Therefore, chromatography that yields overlapping and interfering peaks is much better tolerated. The overall accuracy in quantifying the amount of material in a two-dimensional gel spot is low, so that only large changes in expression (100% or more) of the more abundant proteins are likely to be noted. In contrast, quantitation of changes in the expression rate obtained by measuring the isotope ratio of the analyte is measured with high accuracy (0.1% or less) and over a 1,000-fold range by CRIMS. Thus, according to aspects of the present invention it is possible to obtain not just “more data”, but “more reliable data”.

The present invention, in its various aspects, can address and solve many types of problems found under the category of “functional genomics”. All cellular actions must, in some way, be controlled by genes and their products. Now that the entire DNA sequences of many living organisms are available, attention is moving to understand what information contained in the DNA is transferred to the biological systems and how the information affected the biological systems in terms of structure, function and regulation of control mechanisms. To understand the information transferred from the genome into the biological effector requires an analytical approach that allows the study of biochemical events in a comprehensive way. With the present “comprehensive approach”, biomolecules are not studied “one by one”, but “class by class” or even in their entirety. To describe these methods of analysis, new definitions have been recently coined for each class of materials under investigation. See Veenstra, et al., J. Am. Soc. Mass Spectrom., 11:78-82 (2000). Among the widely accepted names are genomics (comprehensive study of genomic material) and more recently proteomics (comprehensive study of all the proteins in a specific organism). See Oliver, S., Nature, 403 (6770):601-3 (2000). The quantitative isotope ratio-monitoring strategy is truly the analytical conceptualization of these comprehensive studies. This notion is based on the following evidence:

(I) Any biological molecule (regardless of its size or function) contains the same chemical elements (that is, C, O, H) with the inclusion of a few more (e.g., N, S, P) for specific classes of biological molecules (for example, proteins, nucleic acids, etc.);

(II) Each one of these organic elements is always present in different isotopic species (for example, ¹²C, ¹³C, ¹⁴C), and the ratio between the isotopic species of an element is an important physicochemical parameter that can be measured with high accuracy; and

(III) Quantitative isotope ratio-monitoring allows high precision analyses of these common traits (for example, the isotopic ratios of these common elements) shared by any biological molecules regardless of the size, structure or other chemical characteristics thereof.

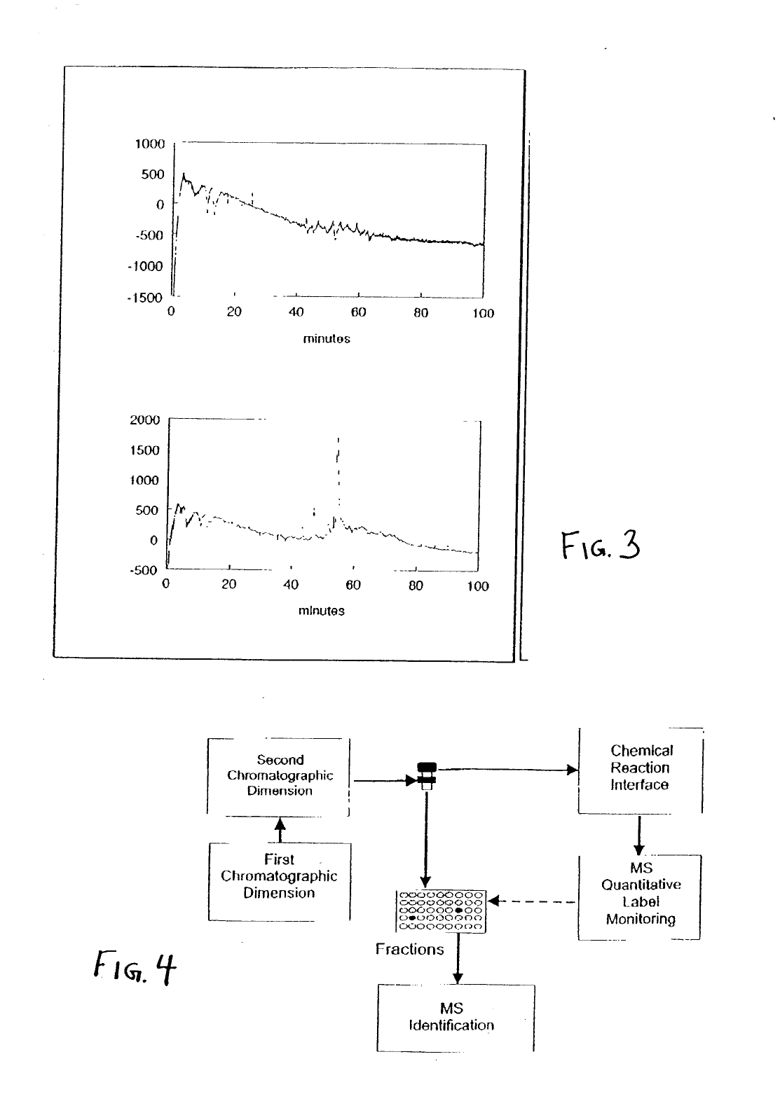

Using the “CRIMS for quantitative differential display” approach, the baseline value for enrichment makes species with similar expression in the two cell populations disappear, and only those species with different expression levels are noted in a chromatographic format (“isotope enrichment trace”). Therefore, defining exactly what species are of interest would greatly reduce the number of samples to be identified by mass spectrometry or by any other analytical method (because only a few of the thousands of species in a given sample are likely to be altered by the experiment). Only specific fractions are used for the identification process instead of the total species extract. CRIMS can be placed on-line with a chromatographic separation procedure. With this configuration, an automated collection method can be provided whereby an altered isotope ratio triggers a sample collector that can then feed into a “high throughput” device to generate a highly efficient system. See FIG. 4.

That is, the isotope ratio-monitoring detector can be used as a “trigger” or “filter” to point to those differentially expressed components that have been collected by splitting the sample, as indicated in FIGS. 1 and 4. The process, illustratively, can be automated by a sensor that is a “gate” to only collect samples when the isotope ratio moved away from a baseline. The use of isotope ratio monitoring to direct the second stage of a two-step structural identification procedure (involving a conventional mass spectrometer or other appropriate structure-determining method) falls within the scope of the present invention.

CRIMS for quantitative isotope ratio-monitoring can be used by itself, to obtain accurate quantitative results, and also assist the “conventional” mass spectrometric identifications of biological molecules by being part of a hybrid instrument. An example of such instrument is given in FIG. 4, which is a schematic of a multidimensional chromatographic separation, followed by a split sending some of the sample to a fraction collector and the rest to CRIMS. In this scheme of FIG. 4, several chromatographic dimensions may be used (if needed) and the sample stream is split. As an example, some sample is deposited automatically into a multiwell plate and some goes to the CRIMS system for label monitoring. Coordination of the labeling with the fractions collected directs the analyst to just those samples (marked in black in FIG. 4) with the desired species.

Similarly, with post-translational modifications, with conventional approaches the analyst somehow has to recognize which species has been modified and examine aspects of the mass spectrum of that protein to understand if that modification is present. With CRIMS, the only target philosophy is asking the CRIMS apparatus to focus its attention on the channel(s) that relate to the anticipated change, and all species having that change are detected and quantified.

Aspects of the present invention can be applied in at least the following areas:

(a) Identification of new pharmacological treatments as well as for the definition of new pharmacological targets by comparing cells with and without pharmacological treatments;

(b) Manifestations of diseases by comparing normal and diseased cells;

(c) Survey of gene-modified cells giving a comprehensive view of what the transformation has accomplished; and

(d) Quality control in the biotechnology industry so that the array of overexpressed proteins can be quantitatively monitored from batch to batch.

Accordingly, the present invention provides a process, and apparatus, using isotope ratio monitoring, together with isotope labeling (e.g., uniform isotope labeling), which allow detection and quantification of substances expressed in different concentrations under different conditions in a general manner, and that does not depend on any operator assumptions about “what to look for”.

What is claimed is:

1. Method for quantitatively monitoring components of a cell population, comprising:
(a) labeling a population of cells with stable isotopes;
(b) mixing the cells labeled with the stable isotopes, with control cells grown without being labeled with the stable isotopes, or which are labeled to a different extent than the cells labeled with the stable isotopes, or which have an isotope difference from the cells labeled with the stable isotopes, so as to form a mixture; and
(c) detecting resulting isotopic enrichment from said mixture, using an isotope ratio-monitoring detector.

2. Method according to claim 1, wherein the population of cells is uniformly labeled with the stable isotopes.

3. Method according to claim 1, wherein said detecting is performed by separating material from the cells to provide a sub-unit, of the cells, under consideration, and measuring the isotopic enrichment of the sub-unit of the cells.

4. Method according to claim 3, wherein said separating is performed by high performance liquid chromatography, and said measuring is performed by chemical reaction interface mass spectrometry.

5. Method according to claim 1, wherein the population of cells are cells within an animal or human, and wherein the labeling is performed by feeding or administering to the animal or human labeled nutrients that become incorporated in a class of biological molecules being studied.

6. Method according to claim 1, wherein said detecting includes subtracting a baseline value from a measured isotopic value, to obtain the resulting isotopic enrichment.

7. Method for quantitatively monitoring a difference between components of first and second samples of respective cell populations growing in different conditions, comprising:
(a) labeling a first population of cells with stable isotopes, forming a labeled first population;
(b) labeling a second population of cells with stable isotopes, and providing a stimulus or variant of the original cells, forming a labeled second population;
(c) combining control cells with the labeled first population, to form the first sample;
(d) combining control cells with the labeled second population, to form the second sample;
(e) detecting resulting isotopic enrichment of the first and second samples, using an isotope ratio-monitoring detector, and comparing the isotopic enrichments of components of the two populations.

8. The method according to claim 7, wherein the detecting includes providing an indication of how much of a difference in isotopic enrichment there is between the first and second samples.

9. The method according to claim 8, wherein the detecting, for each sample, includes separating material of each sample to provide a sub-unit of each sample, of the cells, under consideration, and measuring isotopic enrichment of each sub-unit, prior to said providing the indication of how much of a difference there is between the first and second samples.

10. The method according to claim 7, wherein the detecting, for each sample, includes separating material of each sample to provide a sub-unit, of cells of each sample, under consideration, and measuring the isotopic enrichment of the sub-unit of each sample.

11. The method according to claim 10, wherein the sub-unit is a sub-cellular component.

12. The method according to claim 10, wherein the sub-unit is a class of biological compound.

13. The method according to claim 10, wherein said measuring is performed by isotope ratio monitoring.

14. The method according to claim 13, wherein said isotope ratio monitoring is performed by mass spectrometry.

15. The method according to claim 14, wherein the mass spectrometry is chemical reaction interface mass spectrometry.

16. The method according to claim 10, wherein after said separating, some of the separated material is diverted from said measuring apparatus, for a structural analysis of the material.

17. The method according to claim 7, wherein the measuring includes a monitoring of the isotopic enrichment of each of the first and second samples.

18. The method according to claim 17, wherein the monitoring is a continuous monitoring of the isotopic enrichment of each of the first and second samples.

19. The method according to claim 7, wherein each of the first and second populations of cells is uniformly labeled with the stable isotopes.

20. The method according to claim 7, wherein the detecting includes subtracting a percentage of a measured value of the first sample from a measured value of the second sample, in providing a comparison of the isotopic enrichments.

21. The method according to claim 7, where in the comparing step a difference between the isotopic enrichments of components of the two populations is at least a first value, material of the second sample is collected and transferred for structural analysis of the collected and transferred material.

22. Method for tagging moieties in biological molecules, comprising:
(a) providing a population of cells with an isotopically labeled precursor to provide a population of labeled treated cells;
(b) mixing the treated cells with control cells grown without being provided with the labeled precursor, to provide a mixture of cells; and
(c) detecting isotopic enrichment of the mixture of cells, using an isotope ratio-monitoring detector.

23. The method according to claim 22, wherein said detecting includes initially separating sub-units of the mixture of cells, and measuring isotopic enrichment of a sub-unit, of the sub-units, of the mixture of cells.

24. The method according to claim 23, wherein the measuring isotopic enrichment of the sub-unit is performed by mass spectrometry.

25. The method according to claim 24, wherein the mass spectrometry is chemical reaction interface mass spectrometry.

26. Apparatus for quantitatively monitoring components of a cell population, comprising:
(a) a first structure wherein a population of cells are labeled with stable isotopes, to provide a labeled population;
(b) a mixing structure wherein the labeled population is mixed with control cells grown without being labeled with the stable isotopes, to provide a mixture; and
(c) detecting structure to detect isotopic enrichment of said mixture, said detecting structure including an isotope ratio-monitoring detector.

27. Apparatus according to claim 26, wherein the detecting structure includes a separation structure, for separating sub-units of the mixture, and the isotope ratio-monitoring detector; and the apparatus further includes first transfer structure to transfer sub-units of the mixture from the mixing structure to the separation structure, and second transfer structure for transferring separated sub-units from the separation structure to the isotope ratio-monitoring detector.

28. Apparatus according to claim 27, further comprising an analytical structure to provide structural analysis of the separated sub-units, and third transfer structure for transferring a portion of the separated sub-units from the separation structure to the analytical structure to provide structural analysis, a remaining part of the separated sub-units being transferred by the second transfer structure.

29. Apparatus for providing quantitative differential display between components of first and second samples of respective cell populations growing in different conditions, comprising:
(a) a first structure wherein a first population of cells are labeled with stable isotopes;
(b) a second structure wherein a second population of cells is labeled with said stable isotopes, wherein said second structure includes a first transfer structure to transfer a stimulus or variant to the second structure;
(c) first mixing structure where the first population of cells labeled with the stable isotopes is mixed with unlabeled cells, to provide the first sample;
(d) second mixing structure where the second population of cells labeled with the stable isotopes and having had the stimulus or variant included in the second structure, is mixed with unlabeled cells, to provide the second sample; and
(e) detecting structure to detect isotopic enrichment of each of the first and second samples, the detecting structure including an isotope ratio-monitoring detector.

30. Apparatus according to claim 29, wherein the detecting structure includes separation structure to separate sub-units of each of the first and second samples, and the isotope ratio-monitoring detector to measure isotopic enrichment of the sub-units of each of the first and second samples.

31. Apparatus according to claim 30, wherein the detecting structure further includes comparison structure to compare isotopic enrichment of the sub-units of the first and second samples.
